(19) United States
(12) Patent Application Publication
(10) Pub. No.: US 2003/0027229 A1
(43) Pub. Date: Feb. 6, 2003

(54) Methods of identifying potentially therapeutically selective and effective anti-cancer agents that are inducers of apoptosis

(75) Inventors: Shailaja Kasibhatla, San Diego, CA (US); P. Sanjeeva Reddy, San Diego, CA (US); John A. Drewe, Carlsbad, CA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Cytovia, Inc.

(21) Appl. No.: 10/046,548

(22) Filed: Jan. 16, 2002

Related U.S. Application Data
(60) Provisional application No. 60/294,617, filed on Jun. 1, 2001.

(51) Int. Cl. C12Q 1/68; G01N 33/574

(52) U.S. Cl. 435/7.23; 435/6

(57) ABSTRACT

Disclosed are methods for identifying potential therapeutically effective anti-cancer agents. In particular, the invention relates to the use of biochemical and cell based screening assays to identify compounds that directly or indirectly activate the apoptosis cascade and further a method for identifying those apoptosis inducers that are selective and effective apoptosis agents for use in treating cancer and other therapeutic indications characterized by a lack of appropriate apoptosis. Also disclosed is a method of identifying selective caspase inducers using a cell-line specific primary screen.

### CROSS REFERENCE TO RELATED APPLICATIONS

This application claims the benefit of U.S. Provisional Application No. 60/294,617, filed Jun. 1, 2001.

### BACKGROUND OF THE INVENTION

1. Field of the Invention

This invention relates to a method for identifying potential therapeutically effective anti-cancer agents. In particular, the invention relates to the use of biochemical and cell based screening assays to identify compounds that directly or indirectly activate the apoptosis cascade and further a method for identifying those apoptosis inducers that are selective and effective apoptosis agents for use in treating cancer and other therapeutic indications characterized by a lack of appropriate apoptosis. Also relates to a method of identifying selective caspase inducers using a cell-line specific primary screen.

2. Related Art

Cancer is a group of diseases characterized by the uncontrolled growth and spread of abnormal cells. The occurrence of cancer increases as individuals age, and the majority of cases affect adults who are middle-aged or older. In the U.S., men have a 1 in 2 life time risk of developing cancer, whereas for women the risk is 1 in 3. More than 11 million new cancer cases have been diagnosed since 1990 in the U.S., and more than 1 million new cancer cases are diagnosed each year. Cancer is the second leading cause of death in the U.S., exceeded only by heart disease, and since 1990 there have been approximately 5 million cancer related deaths.

The primary methods of treatment for cancer are surgery, irradiation, and chemotherapy with antineoplastic agents. Progress has been made in enhancing the effectiveness of each of these methods. Thus, curative treatments have been developed for such otherwise fatal cancers as leukemia and testicular cancer. It is equally true that there is still a major need for improvement in virtually every therapeutic method for the treatment of cancer, especially in the case of chemotherapy with antineoplastic agents (Hardman, Limbird, Molinoff, Ruddon and Gilman, Eds., Goodman and Gilman's The Pharmacological Basis of Therapeutics, Ninth Edition, McGraw-Hill, New York, pages 1225-1287 (1996)).

It is remarkable that the earliest antineoplastic agents were actually derived from the war gas “sulfur mustard” first used in World War I. The closely related “nitrogen mustards” were studied somewhat later. The first clinical studies using these compounds were undertaken in 1942, which began the modem use and development of antineoplastic agents. Even today, the nitrogen mustard drugs form a substantial part of the antineoplastic drug inventory. In view of the derivation of antineoplastic agents from war gases, it is not surprising that drug toxicity remains an important limitation of antineoplastic therapy.

An essential element in connection with the drug of a therapeutic agent is its therapeutic index. The therapeutic index is obtained by first determining the median effective dose, also termed the ED₅₀, which is the dose of the drug required to produce a specified effect in 50% of a test animal population. Next, the median lethal dose, or LD₅₀, is obtained by determining the dose that is lethal for 50% of an animal population. The ratio of the LD₅₀ to the ED₅₀ is the therapeutic index, or T.I. It is obviously desirable to use drugs with as high a T.I. value as possible, as these will be the safest drugs for any given purpose.

The T.I. values for the antineoplastic agents are typically among the lowest of any class of medicinal agents. This low T.I. is one of the greatest single problems facing the physician in treating a patient with a neoplastic disease. In addition to considering whether an antineoplastic agent is suited for treating the disease afflicting the patient, the physician must consider the renal (kidney) and hepatic (liver) function of the patient, as well as the general state of health of the patient, and his or her willingness to undergo arduous or painful therapy. Thus, it is clear that much progress remains to be made in developing new, safer, and more effective antineoplastic agents, and more particularly, in methods for the identification of such agents.

An essential question to be answered in attempting to reduce the toxicity of an entire class of therapeutic agents, such as the antineoplastic agents, is the identity of the mechanisms by which the agents exert their therapeutic effect. The goal in this type of analysis is to obtain new agents that act by mechanisms producing fewer toxic effects.

In the case of current antineoplastic drugs, the mechanism of action frequently involves an attack at specific phases of the cell cycle. In brief, the cell cycle refers to the stages through which cells normally progress during their lifetimes. Normally, cells exist in a resting phase termed G₀. During multiplication, cells progress to a stage in which DNA synthesis occurs, termed S. Later, cell division, or mitosis occurs, in a phase called M. Antineoplastic drugs such as cytosine arabinoside, hydroxyurea, 6-mercaptopurine, and methotrexate are S phase specific, whereas antineoplastic drugs such as vincristine, vinblastine, and paclitaxel are M phase specific. Many slow growing tumors, for example colon cancers, exist primarily in the G₀ phase, whereas rapidly proliferating normal tissues, for example bone marrow, exist primarily in the S or M phase. Thus, a drug like 6-mercaptopurine can cause bone marrow toxicity while remaining ineffective for a slow growing tumor. Further aspects of the chemotherapy of neoplastic diseases are known to those skilled in the art (see, e.g., Hardman, Limbird, Molinoff, Ruddon and Gilman, Eds., Goodman and Gilman's The Pharmacological Basis of Therapeutics, Ninth Edition, McGraw-Hill, New York, pages 1225-1287 (1996)).

Because of the severity of the problem of neoplastic disease in a public health sense, as briefly indicated above, an extensive search for antineoplastic drugs has been undertaken in past years in government, academic, and industry laboratories. The National Cancer Institute has sponsored a wide-ranging empirical search for such agents for many years. Testing was undertaken in animals, usually mice, which were inoculated with tumor cell lines. The results of these empirical programs have been limited, but they provided the relatively unsatisfactory drugs that are in use today.

More recently, antimetabolite theory and rational drug design approaches have been used. The Nobel Prize in Medicine or Physiology was awarded to Gertrude Elion and George Hitchings for their extensive studies leading to new antineoplastic antimetabolites. Lengthy x-ray crystallographic studies on thymidilate synthase have led to inhibitors of the enzyme that are useful as antineoplastic agents. Nevertheless, none of these approaches, from the early “brute force” methods to the later elegant crystallographic work has resulted in “breakthrough” antineoplastic drugs.

Today, high throughput screening (HTS) and combinatorial chemistry offer the possibility of screening thousands of compounds, even hundreds of thousands of compounds, in a relatively short time. The availability of automated methods has been an important factor in this development. These methods obviously cannot be applied to whole animal screening, but must be restricted to cellular or macromolecular targets. Thus, a long felt need exists for assay methods suitable for the HTS discovery of effective new antineoplastic agents.

Prior cell based assays involved treatment of cell lines for prolonged periods with potential antineoplastic drugs. For example, the National Cancer Institute screen involves a panel of 60 cell lines that are treated for a prolonged period, for example 48-60 hours. The control cells are then counted and compared to the number of cell colonies in the treated samples. These methods, which result in dead or dying cells, have been disappointing in that they have failed to identify drugs that are effective in the presence of tumor lines with mutated p53 phenotypes.

A normal checkpoint in the life of cells in multicellular organisms is the process of apoptosis (see, e.g., Evan and Littlewood, Science 281:1317-1322 (1998). Apoptosis is the highly conserved mechanism by which cells commit suicide. Characteristics of the process include an execution phase that includes loss of cell volume, plasma membrane blebbing and chromatin condensation, followed by packing of the cellular contents into membrane-enclosed vesicles called apoptotic bodies that are rapidly phagocytosed by neighboring cells.

Apoptosis is one of several mechanisms that cells employ in response to the hazards raised by DNA damage. In particular, apoptosis is an effective action for damaged cells that can replicate and become cancerous. One major mediator of apoptosis is p53, which is a transcription factor that is normally present in low amounts because it is subject to a destruction process signaled by the Mdm-2 protein. Additionally, p53 can induce growth arrest in cells. In human cancer, p53 is often functionally inactivated so that its growth arrest activity or apoptotic activity is diminished. Thus, a therapeutic treatment for cancer, in which normal apoptosis is diminished, would be to enhance the apoptotic process through the administration of appropriate drugs. Moreover, since autoimmune disease and certain degenerative diseases also involve the proliferation of abnormal cells, therapeutic treatment for these diseases could also involve the enhancement of the apoptotic process through the administration of appropriate drugs.

It is pertinent, therefore, to inquire into the mechanism of apoptosis in order to develop a method for the identification of such antineoplastic and related drugs. It has been found that a group of proteases are a key element in apoptosis (see, e.g. Thornberry, Chemistry and Biology 5:R97-R103 (1998); Thornberry, British Med. Bull. 53:478-490 (1996)). Genetic studies in the nematode Caenorhabditis elegans revealed that apoptotic cell death involves at least 14 genes, two of which are the pro-apoptotic (death-promoting) ced (for cell death abnormal) genes, ced-3 and ced-4. CED-3 is homologous to interleukin 1β-converting enzyme, a cysteine protease, which is now called caspase-1. When these data were ultimately applied to mammals, and upon further extensive investigation, it was found that the mammalian apoptosis system appears to involve a cascade of caspases, or a system that behaves like a cascade of caspases. At present, the caspase family of cysteine proteases comprises 10 different members, and more may be discovered in the future. All known caspases are synthesized as zymogens that require cleavage at an aspartyl residue prior to forming the active enzyme. Thus, caspases are capable of activating other caspases, in the manner of an amplifying cascade.

The caspase cascade can be involved in disease processes in two major aspects. Excessive activity of the caspase cascade can lead to excessive apoptosis and organ failure. Among the diseases that could result from this excessive activity are myocardial infarction, congestive heart failure, viral infections, rheumatoid arthritis and others. Inhibitors of the caspase cascade could thus be candidates for therapeutic intervention in such diseases. Inasmuch as methods for the discovery of enzyme inhibitors is a frequently practiced art, numerous approaches to the discovery of caspase inhibitors are available (see Villa et al., Trends Biochem Sci. 22:388-393, (1997); Liang and Fesik,. Mol. Biol. 274:291-302 (1997)).

Although the development of enzyme inhibitors as therapeutic agents is well understood art (see Muscate and Kenyon, Burger's Medicinal Chemistry 1:733-782, ₅^{th} Ed. (1995) this is not the case in the development of enzyme activators. The theoretical basis for the development of enzyme activators is still in its infancy. In the case of the apoptosis process, control points are known to exist that represent points for intervention leading to activation. These control points include the CED-9-BCL-like and CED-3-ICE-like gene family products, which are intrinsic proteins regulating the decision of a cell to survive or die and executing part of the cell death process itself, respectively (see Schmitt et al., Biochem. Cell. Biol., 75:301-314 (1997)). BCL-like proteins include BCL-xL and BAX-α, which appear to function upstream of caspase activation. BCL-xL appears to prevent activation of the apoptotic protease cascade, whereas BAX-α accelerates activation of the apoptotic protease cascade. Thus, it is clear that the possibility exists for the activation of the caspase cascade, although the exact mechanisms for doing so are not clear at this point. Inasmuch as it is equally clear that insufficient activity of the caspase cascade and consequent apoptotic events are implicated in various types of cancer and possibly in various degenerative and autoimmune diseases, the development of caspase cascade activators is a highly desirable goal in the development of potentially therapeutically effective antineoplastic agents.

In order to identify caspase cascade activators that are potentially therapeutically effective antineoplastic agents, a number of issues must be resolved that have not been adequately addressed by the prior art. First, inasmuch as about half of all cancers have mutations of p53, it is important to use a method that activates the caspase cascade directly or indirectly, independently of p53. This means that an isolated enzyme assay, wherein the enzyme is an individually isolated caspase, or a caspase assay in dead cells (see Los et al., Blood, 90:3118-3129, (1997)) is unsuitable because receptors and/or cofactors that influence the caspase cascade might not be present in such preparations. In addition, because the caspase cascade is intracellular, compounds that influence it must ordinarily cross the cell membrane, in contrast to many other types of drugs that interact with the external elements of transmembrane receptors. Thus, an assay in viable cells having an intact cell membrane must be used for the present purpose. Thirdly, any assay for this purpose should be amenable to a high-throughput mode embodiment if it is to be useful for broadly based drug screening. Thus, it should be an assay that can be carried out quickly.

However, there are substantial technical obstacles to the development of cellular assays for caspase activators. Prior attempts to make the discovery of potentially therapeutically effective antineoplastic agents that act as caspase activators more accurate, cost effective, and rapid, have not achieved the simplicity and reliability of operation of the present invention.

Identification of compounds that modulate caspase cascade activity, such as those that act as specific activators, may not only aid in elucidating the function of caspases, but may yield therapeutically useful compounds. In particular, compounds that specifically activate specific elements of the caspase cascade should be useful in identifying essential characteristics of those elements and should aid in the design of therapeutic disease specific agents.

Different malignancies result from defects at various levels that eventually converge in the central mechanism of defective apoptosis. As a result the tumor cell by virtue of its lack of apoptosis ends up into a malignancy. Therefore, the present invention provides a method for identifying compounds that activate the caspase cascade taking advantage of the differences in the anti-apoptotic mechanisms that prevail in the various tumors. To this effect the invention is directed into the use of the primary tumor cells or any tumor cell line originated from different tissue origin to be used in the primary screening assay.

Since death is central to the function of any cell, the central apoptotic mechanism will also be common to all the cells. However, the signaling pathways that lead to the final activation of the apoptosis cascade could be different in the various tumors. For example, even though caspase activation is the end point read-out for apoptosis, these caspases could be activated at the level of the enzyme, or through a signaling pathway either mediated through the death receptor or the mitochondria, or any other indirect mechanisms involving the proliferative or growth signaling pathways that finally lead to apoptosis. Therefore, the present invention is also directed toward the identification of criteria for a caspase inducer to be effective as a selective and effective apoptosis agent only in the targeted cell, tissue or organ. The present invention is also directed to the use of compounds that act as activators of the caspase cascade in viable cultured eukaryotic cells having an intact cell membrane. Such compounds should be useful as therapeutic agents for the treatment of caspase cascade-mediated diseases and disorders, such as neoplastic diseases and disorders, and also for the identification of the function of the caspase cascade.

### SUMMARY OF THE INVENTION

The present invention is directed to methods for identifying direct and indirect activators of the apoptosis cascade, therapeutic methods to use such activators, compositions for such activators, and kits comprising such activators.

More particularly, the invention relates to a method for identifying potentially therapeutically effective anti-cancer agents by determining the ability of one or more test compounds to selectively and differentially activate the apoptosis cascade in viable cultured cancer cells having an intact cell membrane when the cells are exposed to the test compound(s) for a predetermined period of time at a predetermined temperature, wherein a test compound is determined to have potential therapeutic efficacy if said caspase cascade activity is enhanced in response to the presence of said test compound.

In particular, the invention relates to a method for identifying selective anticancer agents by determining the ability of one or more test compounds to activate the apoptosis cascade in a first type of viable cultured cancer cells having an intact cell membrane when the cells are exposed to test compound(s) for a predetermined period of time at a predetermined temperature, wherein a test compound is determined to have potential therapeutic efficacy if said caspase cascade activity is enhanced in response to the presence of said test compound, and comparing the result to that obtained with at least one other type of viable cultured cancer cells having an intact membrane, wherein when a test agent exhibits enhanced caspase cascade activity in one or more types of cancer cells compared to at least one other type of cancer cells, it is identified as a selective anticancer agent.

More particularly, the invention relates to a method for identifying selective anticancer agents comprising obtaining at least one population of viable cultured cancer cells of a first type having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; combining a second portion of the at least one population with an amount of the solvent which was used to dissolve the at least one test compound, for the predetermined period of time at the predetermined temperature thereby generating a second volume; separately adding to each of the first volume and the second volume a reporter compound having at least one measurable property which is responsive to the caspase cascade; measuring the at least one measurable property of the reporter compound in the first volume and thereby measuring the caspase cascade activity of the first volume; measuring the at least one measurable property of the reporter compound in the second volume and thereby measuring the caspase cascade activity of the second volume; calculating a first ratio of caspase cascade activity measured for the first volume to the caspase cascade activity measured for the second volume; and comparing the first ratio to at least one second ratio obtained with at least one second type of cultured cancer cells, and identifying those test compounds that have higher ratios for certain types of cultured cancer cells and are selective therefor.

In this embodiment, the results using different cancer cells tested separately are compared to identify anticancer agents that are selective for one or more particular cancers. This may be carried out by comparing the calculated ratios and identifying those test compounds having the highest ratio for particular cancer cells. Alternatively or additionally, the selectivity may be determined by calculating the concentration of test compound necessary to give a 50% inhibition of growth (GI₅₀) of a first cell type and comparing that GI₅₀ to the GI₅₀ in at least one other cell type. Test compounds with lower GI₅₀s in one or more particular cell types are selective therefor.

The invention further relates to a method for confirming the initial results obtained according to the invention by determining the cancer cell viability with or without the test compound to confirm that the test compound selectively kills the cancer cell. In this embodiment, the invention relates to a further method for identifying a selective anti-cancer agent, comprising obtaining at least one population of viable cultured cancer cells having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; combining a second portion of the at least one population with an amount of the solvent which was used to dissolve the at least one test compound, for the predetermined period of time at the predetermined temperature thereby generating a second volume; separately assessing the cell viability of the first volume and the second volume; and comparing the cell viability of the first volume to the cell viability of the second volume, wherein when the cell viability of the first volume is less than the cell viability of the second volume, the at least one test compound selectively kills the cancer cells and is identified as a selective anti-cancer agent.

The invention further relates to a method to further determine the specificity of anticancer agents by determining the ability of the agent to arrest the cell cycle during a particular phase prior to apoptosis, comprising obtaining at least one population of viable cultured cancer cells having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; and determining at what phase the cell cycle is arrested.

The invention further relates to a method to further determine the specificity of anticancer agents by determining the ability of the agent to inhibit tubulin polymerization. Preferably, the method comprises contacting a first sample of tubulin with a predetermined amount of at least one test compound dissolved in a solvent for a predetermined period of time at a predetermined temperature thereby generating a first volume; separately combining a second sample of tubulin with an amount of the solvent which was used to dissolve the at least one test compound, for the predetermined period of time at the predetermined temperature thereby generating a second volume; and comparing the extent of polymerization of the first and second samples of tubulin and thereby determining wherein the test compound inhibits the polymerization of tubulin.

### BRIEF DESCRIPTION OF THE FIGURES

FIG. 1A depicts a plot of the DNA content of DMSO treated cells.

FIG. 1B depicts a plot of the DNA content of test compound treated cells.

FIG. 2A depicts a plot of DNA content of DMSO treated cells indicating the different cell cycle regions.

FIG. 2B depicts a plot of DNA content of test compound treated cells showing an increase in SubG1 apoptotic cells.

FIG. 3 depicts a graph of OD₃₄₀ vs. time in a tubilin polymerization assay in the presence of control, test compound 2105, paclitaxel and nocodazole.

FIG. 4 is a scatterplot showing the results of a screening assay with T47D cells and activated T cells.

FIG. 5 is a scatterplot showing the results of a screening assay with T47D cells and HL-60 cells.

FIG. 6 is a scatterplot showing the results of a screening assay with T47D cells and Jurkat cells.

FIG. 7 is a scatterplot showing the results of a screening assay with Jurkat cells and HL-60 cells.

### DETAILED DESCRIPTION OF THE INVENTION

Definitions

Unless defined otherwise, all technical and scientific terms used herein have the same meaning as is commonly understood by one of ordinary skill in the art to which this invention belongs.

As used herein, apoptosis is a highly conserved, genetically programmed form of cellular suicide characterized by distinct morphological changes such as cytoskeletal disruption, cell shrinkage, membrane blebbing, nuclear condensation, fragmentation of DNA, and loss of mitochondrial function.

As used herein, a caspase is a cysteine protease of the interleukin-1β/CED-3 family. As used herein, the caspase cascade is a sequential activation of at least two caspases, or the activation of caspase activity that behaves as if it involves the sequential activation of at least two caspases.

As used herein an effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

As used herein, an activator of the caspase cascade is a compound, such as a drug or antibody, that enhances caspase-mediated physiological responses such as cellular apoptosis. The activator may act by any one or a combination of mechanisms.

As used herein, pharmaceutically acceptable salts or prodrugs of the compounds include any salts, esters or derivatives that may be readily prepared by those of skill in this art using known methods for such derivatization and that produce compounds that may be administered to animals or humans without substantial toxic effects and that either are pharmaceutically active or are prodrugs. For example, hydroxyl groups can be esterified (e.g., with a C₂₋₁₀ alkanoyl group or succinic acid) or etherified (with a C₁₋₆ alkoxy methylchloride). In addition, a carboxylic acid group may be esterified (e.g. with a C₁₋₆ alcohol). Examples of such salts include the acid addition salts of amino compounds. Such salts include the chloride, sulfate, hemisulfate, phosphate, hydrogen phosphate, dihydrogen phosphate, acetate, maleate, succinate, and the like. Other salts include the salts of carboxy-containing compounds which may be prepared by reacting the carboxy-containing compound with a base such as sodium hydroxide, lithium hydroxide, potassium hydroxide, sodium bicarbonate, sodium carbonate, and the like.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady, Medicinal Chemistry: A Biochemical Approach, Oxford University Press, New York, pages 388-392 (1985)). For example, succinylsulfathiazole is a prodrug of 4-amino-N-(2-thiazoyl)benzenesulfonamide (sulfathiazole) that exhibits altered transport characteristics.

As used herein, treatment means any manner in which the symptoms of a condition, disorder or disease are ameliorated or otherwise beneficially altered.

Animals which may be treated according to the present invention include all animals which may benefit from the administration of an anticancer agent identified according to the present invention. Such animals include mammals such as humans, cows, pigs, sheep, dogs, cats, horses, and the like.

As used herein, amelioration of the symptoms of a particular disorder by administration of a particular pharmaceutical composition refers to any lessening, whether permanent or temporary, lasting or transient, that can be attributed to or associated with administration of the composition.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, may be a mixture of stereoisomers. In such instances, further purification might increase the specific activity of the compound.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions, and mixtures.

As used herein, a fluorogenic, chromogenic or chemiluminescent substrate is a substance that produces fluorescence, light absorption within the ultraviolet, visible or infrared spectrum, or light emission under the influence of the caspase cascade.

Identifying Compounds That Activate the Caspase Cascade

The test compounds may be pure substances or mixtures of substances such as in combinatorial libraries. The test compounds may be any natural product, synthesized organic or inorganic molecule, or biological macromolecules. Preferably, the test compounds are preselected to have <500 MW, <5 II-bond donors, <II-bond acceptors, and logP<5. Computer programs may be used to diversify the compound library. Preferably, the test compounds are at least 85% pure.

The reporter molecule is composed of at least two covalently linked parts. One part is an amino acid sequence which may be recognized by any of the intracellular proteases or peptidases that are produced as a result of caspase cascade activation. This sequence is bonded to an aromatic or conjugated moiety that undergoes a detectable physical change upon its release from all or part of the amino acid sequence. Such moieties include a fluorogenic moiety that fluoresces more strongly after the reporter molecule is hydrolyzed by one of the proteases, a chromogenic moiety that changes its light absorption characteristics after the reporter molecule is hydrolyzed by one of the proteases, or a chemiluminescent moiety that produces light emission after the reporter molecule is hydrolyzed by one of the proteases. Alternatively, the aromatic or conjugated moiety may be linked to a plurality of aminoacid sequences.

One type of such a reporter molecule is given by Formula I:

x-y-z (I)

or biologically acceptable salts or pro-reporter molecules (such as methyl ester form of carboxyl-containing amino acid residues) thereof, wherein x and z is the same or different and is a peptide or amino acid or acyl group or other structure such that compounds of Formula I are substrates for a caspase or other enzyme involved in the intracellular apoptosis cascade; and wherein the scissile bond is only one or both of the x-y and y-z bonds in Formula I when x is the same as z, or wherein the scissile bond is only one of the x-y or y-z bond in Formula I when x is not the same as z, y is a fluorogenic or fluorescent moiety. See International Application WO 98/18856.

Preferred compounds are represented by Formula II:

R₁-(AA)ₙ-ASp-y-ASp-(AA)ₙ-R₁ (II)

or biologically acceptable salts or pro-reporter molecules (such as methyl ester form of carboxyl-containing amino acid residues) thereof, wherein R₁ is an N-terminal protecting group such as t-butyloxycarbonyl, acetyl, and benzyloxycarbonyl; each AA independently is a residue of any natural or non-natural α-amino acid or β-amino acid, or derivatives of an α-amino acid or β-amino acid; each n is independently 0-5; and y is a fluorogenic or fluorescent moiety. Preferred y is a Rhodamine including Rhodamine 110, Rhodamine 116 and Rhodamine 19. Most preferred y is Rhodamine 110.

Especially preferred compounds are represented by Formula III:

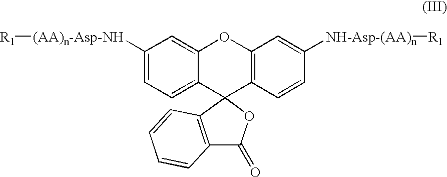

or biologically acceptable salts or pro-reporter molecules (such as methyl ester form of carboxyl-containing amino acid residues) thereof, wherein R₁, AA, n are as defined previously in Formula II. Preferred R₁ is t-butyloxycarbonyl, acetyl and benzyloxycarbonyl. Preferred values of n are 1-3.

Another group of preferred compounds falling within the scope of Formula I include compounds wherein x is not the same as z. Preferred compounds of this group include those wherein x is a peptide or other structure which makes the compound a substrate for a caspase or other enzyme related to apoptosis, and the x-y bond in Formula I is the only bond which is scissile under biological conditions. z is a blocking group and the y-z bond in Formula I is not a scissile bond under biological conditions.

Specifically, the fluorogenic or fluorescent reporter compounds that may be used in this invention are of Formula V:

R₁-(AA)ₙ-Asp-y-R₆ (V)

or biologically acceptable salts or pro-reporter molecules (such as methyl ester form of carboxyl-containing amino acid residues) thereof, wherein:

R₁, AA, n and y are as defined previously in Formula II; and

R₆ is a blocking group which is not an amino acid or a derivative of an amino acid.

Preferred R₆ blocking groups include, but are not limited to, an alkyloxycarbonyl group such as methoxycarbonyl, an arylalkyloxycarbonyl group such as benzyloxycarbonyl, a C₂₋₆ acyl (alkanoyl) group such as acetyl, a carbamyl group such as dimethylcarbamyl, and an alkyl, haloalkyl or aralkyl sulfonyl group such as methanesulfonyl. Preferred y is a Rhodamine including Rhodamine 110, Rhodamine 116 and Rhodamine 19. Most preferred y is Rhodamine 110.

In particular, preferred embodiments of the compounds of Formula V are represented by Formula VII:

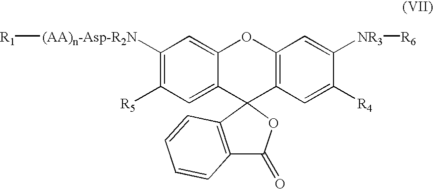

or biologically acceptable salts or pro-reporter molecules (such as methyl ester form of carboxyl-containing amino acid residues) thereof, wherein

R₁, R₆, AA and n are as defined previously in Formulae II and V;

R₂ and R₃ are the same or different and are independently hydrogen, alkyl or aryl; and

R₄ and R₅ are the same or different and are independently hydrogen or alkyl.

Preferred R₁ is t-butyloxycarbonyl, acetyl and benzyloxycarbonyl. Preferred n is 1-3. Preferred R₂ and R₃ are hydrogen, methyl or ethyl. Preferred R₄ and R₅ are hydrogen or methyl. Preferred R₆ blocking groups include, but are not limited to, an alkyloxycarbonyl group such as methoxycarbonyl, an arylalkyloxycarbonyl group such as benzyloxycarbonyl, an acyl group such as acetyl, a carbamyl group such as dimethylcarbamyl, and an alkyl, haloalkyl or aralkyl sulfonyl group such as methanesulfonyl.

Other fluorogenic substrates useful in the practice of the present invention are disclosed in the following U.S. Pat. Nos.: 4,336,186; 4,557,862; 4,640,893; 5,208,148; 5,227,487; 5,362,628; 5,443,986; 5,556,992; 5,587,490; 5,605,809; 5,698,411; 5,714,342; 5,733,719; 5,776,720, 5,849,513; 5,871,946; 5,897,992; 5,908,750; 5,976,822. Useful substrates are also described in EP 0285179 B1; EP 623599 A1; WO 93/04192; WO 93/10461; WO 96/20721; WO 96/36729; WO 98/57664; Ganesh, S. et al., Cytometry 20:334-340 (1995); Haugland, R. and Johnson, I., J. Fluorescence 3:119-127 (1993); Haugland, R., Biotechnic and Histochemistry 70:243-251 (1995); Haugland, R., Molecular Probes Handbook of Fluorescent Probes and Research Chemicals, pp. 28 and 54, 6th Ed. (1996); Holskin, B., et al., Anal. Biochem. 226:148-155 (1995); Johnson, A., et al., Anal. Chem. 65:2352-2359 (1993); Klingel, S., et al., Methods in Cell Biology 41:449-459 (1994); Leytus, S., et al., Biochem. J. 215:253-260 (1983); Leytus, S., et al., Biochem. J. 209:299-307 (1983); Matayoshi, E., et al., Science 247:954-958 (1990); Morliere, P., et al., Biochem. Biophys. Res. Commun. 146:107-113 (1987); O'Boyle, D., et al., Virology 236:338-347 (1997); Richards, A., et al., J. Biol. Chem. 265:7733-7736 (1990); Rothe, G., et al., Biol. Chem. Hoppe-Seyler 373:547-554 (1992); Stevens, J., et al., Eur. J. Biochem. 226:361-367 (1994); Tamburini, P., et al., Anal. Biochem. 186:363-368 (1990); Thornberry, N., et al., J. Biol. Chem. 272:17907-17911 (1997); Toth, M. and Marshall, G., Int. J. Peptide Protein Res. 36:544-550 (1990); Tyagi, S. and Carter, C., Anal. Biochem. 200:143-148 (1992); Weber, J. “Adenovirus Endopeptidase and Its Role in Virus Infection” in The Molecular Repertoir of Adenoviruses 1, Doerfler, W. and Bohm, P. eds., pp. 227-235, Springer Press, New York (1995); Zhang, R., et al., J. Virology 71:6208-6213 (1997); Mangel, W., et al., Biol. Chem. Hoppe-Seyler 373:433-440 (1992); Bonneau, P., et al., Anal. Biochem. 255:59-65 (1998); and DiIanni, C., et al., J. Biol. Chem. 268:25449-25454 (1993).

A caspase substrate reporter molecule is used to determine caspase activation and apoptosis in the cells. However, inasmuch as the caspase cascade takes place in the intracellular environment, measures may be undertaken to enhance transfer of the reporter compound across the cell membrane. This can be accomplished with a suitable permeabilization agent. Preferable permeabilization agents include, but are not limited to, NP-40, n-octyl-O-D-glucopyranoside, n-octyl-O-D-thioglucopyranoside, taurocholic acid, digitonin, CHAPS, lysolecithin, dimethyldecylphosphine oxide (APO-10), dimethyldodecylphosphine oxide (APO-12), N,N-bis-(3-D-gluconamidopropyl)cholamide (Big Chap), N,N-bis-(3-D-gluconamidopropyl)deoxycholamide (Big Chap, deoxy), BRIG-35, hexaethyleneglycol (C10E6), C10E8, C12E6, C12E8, C12E9, cyclohexyl-n-ethyl-O-D-maltoside, cyclohexyl-n-hexyl-O-D-maltoside, cyclohexyl-n-methyl-O-D-maltoside, polyethylene glycol lauryl ether (Genapol C-100), polyethylene glycol dodecyl ether (Genapol X-80), polyoxyethylene isotridecyl ether (Genapol X-100), n-decanoylsucrose, n-decyl-O-D-glucopyranoside, n-decyl-O-D-maltopyranoside, n-decyl-O-D-thiomaltoside, n-dodecanoylsucrose, n-dodecyl-O-D-glucopyranoside, n-dodecyl-O-D-maltoside, n-heptyl-O-D-glucopyranoside, n-heptyl-O-D-thioglucopyranoside, n-hexyl-O-D-glucopyranoside, n-nonyl-O-D-glucopyranoside, n-octanoylsucrose, n-octyl-O-D-maltopyranoside, n-undecyl-O-D-maltoside, n-octanoyl-O-D-glucosylamine (NOGA), PLURONIC® F-127, PLURONIC® F-68, and dimethyl sulfoxide (DMSO).

Cancer cell lines which can be used in the practice of the invention include, inter alia, T47-D, HeLa, PC-3, Panc-1, ZR-75-1, LnCap, K562, DLD-1, A549, and SHP77. Thus, when a test compound is determined to be selective for a breast cancer cell line such as T47D or ZR-75-1, it will be effective for the treatment of the breast cancer in vivo. When a test compound is determined to be selective for a prostate cancer cell line such as PC-3, it will be effective for the treatment of the prostate cancer in vivo.

Preferably, the test compound is incubated with the cell for a predetermined time to allow transport of the test compound across the cell membrane or interaction of the test compound with receptors on the surface of the cell membrane. The predetermined period of time may be about 1 minute to about 48 hours, preferably about 1-24 hours, and most preferably about 3, 5, or 24 hours. The predetermined temperature may be about 4° C. to about 42° C., preferably about 37° C.

The predetermined period of time used in the present invention is sufficiently short to maintain an intact cell membrane in the cells being used in the assay, and makes possible the specificity of the present method for activators of the caspase cascade that are anticancer agents, rather than nonspecific cell poisons. The intactness of the cell membrane may be confirmed by use of propidium iodide (available from Aldrich Chemical Co.). Tested agents found to be active may be confirmed and tested for specificity by testing with various dividing and resting cell types of different tissue or organ origin.

In a preferred embodiment, the test compounds are screened in a high throughput format, e.g. in a 384-well format. Up to 30 plates per day and more may be used to screen 10,560 test compounds 3-4 days/week. Automated procedures may be used in the primary caspase assay including hit picking. The hits are then confirmed in one of the secondary screens discussed below. Compounds identified according to the present invention are useful for treating, preventing or ameliorating cancers such as Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

Measuring the Potency of Caspase Cascade Activation

Using a fluorescent plate reader, an initial reading (T=0) is made immediately after addition of the reporter reagent solution, employing excitation and emission at an appropriate wavelength (preferably excitation at 485 nm and emission at 530 nm) to determine the background absorption and/or fluorescence of the control sample. After the incubation, the absorption and/or fluorescence of the sample is measured as above (e.g., at T=1 hr).

Sample Calculation: Measured relative fluorescence unit (RFU) values are used to calculate the potency of the test compounds. Equation (1) affords determination of the time dependent change in fluorescence of cancer cells both in the presence and absence of a test compound:

RFU_{(T=1 hr)}−RFU_{(T=0)=Net} RFU (1)

If some particular test compound serves directly or indirectly as a caspase cascade activator (i.e., induces apoptosis), then the apoptosis proteases present will cleave the fluorescent substrate resulting in an increase in fluorescence.

Determining the efficacy of an anticancer agent that selectively kills cancer cells may be accomplished by using the following ratio:

$$\frac{\text{Net} \text{RFU} \text{of test compound}}{\text{Net} \text{RFU} \text{of control sample}} = \text{Ratio} \qquad ( 2 )$$

where the numerator represents the Net RFU observed when the cancer cells are in the presence of the test compound; and, the denominator represents the NET RFU observed when none of the anticancer agent is present. Preferred test compounds are those indicating a ratio of 2.0 or greater and most preferably with a measured ratio greater than a statistically significant value calculated as:

(Average Control RFU+(4×SD_{control}))/(Average Control RFU) (3)

wherein SD refers to the standard deviation.

Secondary Screens

The invention further relates to a method for confirming the initial results regarding the potency of caspase cascade activation by determining the cancer cell viability with or without the test compound to confirm that the test compound selectively kills the cancer cell.

In this embodiment, the results with test compounds may be compared to the results obtained with known anticancer agents, antibiotics, immunosuppressives and cell poisons.

In this embodiment, the invention relates to a further method for identifying a selective anti-cancer agent, comprising obtaining at least one population of viable cultured cancer cells having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; combining a second portion of the at least one population with an amount of the solvent which was used to dissolve the at least one test compound, for the predetermined period of time at the predetermined temperature thereby generating a second volume; separately assessing the cell viability of the first volume and the second volume; and comparing the cell viability of the first volume to the cell viability of the second volume, wherein when the cell viability of the first volume is less than the cell viability of the second volume, the at least one test compound selectively kills the cancer cells and is identified as a selective anti-cancer agent.

Cell viability may be assessed by observing mitochodrial activity, membrane intactness, or cell number. Mitochondrial activity, membrane intactness, or cell number may be measured by using fluorescence methodology, colorimetric assays, or direct visualization techniques, and by using a reporter compound selected from the group consisting of a fluorogenic compound that produces fluorescence under the influence changes in mitochondrial activity, membrane intactness, or cell number; a chromogenic compound that produces light absorption under the influence of changes in mitochondrial activity, membrane intactness, or cell number; and a chemiluminescent compound that produces light emission under the influence of changes in mitochondrial activity, membrane intactness, or cell number.

The predetermined period of time may be about 1 minute to about 48 hours, preferably about 1-24 hours, and most preferably about 3, 5, or 24 hours. The predetermined temperature may be about 4° C. to about 42° C., preferably about 37° C.

The invention further relates to a method to further determine the specificity of anticancer agents by determining the ability of the agent to arrest the cell cycle during a particular phase prior to apoptosis. In this embodiment, a timecourse of test compound treatment determines the phase of the cell cycle arrest that precedes apoptosis. The G2M, S/G2M and G1 phases are the major phases in the cell cycle when one cell divides to become two daughter cells.

The cycle starts from a resting quiescent cell (G0 phase) which is stimulated by growth factors leading to a decision (G1 phase) to replicate its DNA. Once the decision is made, the cell starts replicating its DNA (S-phase) and then into a G2 phase before finally dividing into two daughter cells. Cells which then undergo apoptosis contain fragmented DNA in amounts that are less that in the G1 phase and hence are called sub-G1. Thus, a compound leading to a G1 or G2M or S phase arrest and no apoptosis at 24 hr treatment, and leading to apoptosis at 48 hr treatment as determined by the presence of a sub-G1 peak, indicates that the test compound arrest the cell cycle at the respective stage before inducing apoptosis. See Sherr, C. J., Cancer Res. 60:3689-3695 (2000), for a discussion of cancer cell cycles.

Preferably, this aspect of the invention comprises obtaining at least one population of viable cultured cancer cells having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; and determining whether the cell cycle is arrested and at what phase.

In this embodiment, the cells are incubated with a range of concentrations of test compound (e.g. 0.02 μM to 5 μM) for 6 h under normal growth conditions and control cultures are treated with DMSO vehicle. The cells are then treated e.g. for 20 min with 800 nM Syto 16. Cytospin preparations are then prepared and the samples are viewed by fluorescent microscopy using a fluorescein filter set. For each concentration of test compound, the number of mitotic figures are counted and expressed as a percentage of the total number of cells. Three fields from each condition are evaluated and the mean and SEM is calculated and plotted as a function of drug concentration. Another method is to simply stain the nuclei with Propidium Iodide and analyse the DNA content using a Fluorescence Activated Cell Sorter and Cell Quest Software (Becton Dickinson).

The invention further relates to a method to further determine the specificity of anticancer agents by determining the ability of the agent to inhibit or enhance tubulin polymerization. Tubulin is composed of a heterodimer of two closely related 55K dalton proteins called alpha and beta tubulin. These two proteins are encoded by separate genes, or small gene families, whose sequences are highly conserved throughout the eukaryotic kingdom. Consequently, tubulin isolated from bovine brain tissue is highly homologous to tubulin isolated from any eukaryotic source. (Paull, K. D.;

Lin, C. M.; Malspeis, L.; Hamel, E. Cancer Res. 1992, 52, 3892-3900). Thus, tubulin isolated from any eukaryotic source may be used in the practice of the invention.

Tubulin has been a drug target for anticancer compounds as the polymerization of tubulin into microtubules is a critical step in the mitosis (M) phase of the cell cycle that leads to a symmetric and efficient division of the one-cell contents into two of the daughter cells. Tubulin polymerizes to form structures called microtubules (MTs). When tubulin polymerizes it initially forms protofilaments, microtubules consist of 13 protofilaments and are 25 nm in diameter, each mm of microtubule length being composed of 1650 heterodimers. Microtubules are highly ordered fibers that have an intrinsic polarity. Tubulin can polymerize from both ends in vitro, however, the rate of polymerization is not equal. It has therefore become the convention to call the rapidly polymerizing end the plus-end of a microtubule and the slowly polymerizing end the minus-end. In vivo the plus end of a microtubule is distal to the microtubule organizing center.

The ability of tubulin to polymerize into microtubules can be followed very simply by observing an increase in optical density of a tubulin solution at OD340 nm. Thus, according to the present invention, tubulin (see Examples; Paull, K. D.; Lin, C. M.; Malspeis, L.; Hamel, E. Cancer Res. 1992, 52, 3892-3900) is combined with an appropriate buffer (see Examples) and the time coarse of polymerization is monitored using a plate reader and measuring optical density.

A test compound that inhibits the polymerization reaction is a destabilizer (such as Vinblastin). A test compound that increases the polymerization rate is classified as a stabilizer (such as Taxol). Tubulin stabilizers have associated toxicity problems. Thus, according to the present invention, one may identify novel caspase inducers that are tubulin inhibitors or stabilizers..

Preferably, the method comprises contacting a first sample of tubulin with a predetermined amount of at least one test compound dissolved in a solvent for a predetermined period of time at a predetermined temperature thereby generating a first volume; separately combining a second sample of tubulin with an amount of the solvent which was used to dissolve the at least one test compound, for the predetermined period of time at the predetermined temperature thereby generating a second volume; and comparing the extent of polymerization of the first and second samples of tubulin and thereby determining wherein the test compound inhibits or enhances the polymerization of tubulin.

The predetermined period of time may be about 20 min to about 60 min, most preferably about 40-60 min. The absorbance at 340 nm is then plotted against time.

Once a compound has been identified as an anticancer agent according to the present invention, the compound may be further tested and confirmed in animals and, in particular, in animal models of human cancers. Finally, the agents may be tested for efficacy in humans.

The following examples demonstrate usefulness of the invention in measuring the activity of caspases and other enzymes involved in apoptosis in cells and tissues. The examples also demonstrate usefulness of the invention in drug screening assays that can be utilized to find anticancer agents. These examples are illustrative, but not limiting, of the method and compositions of the present invention. Other suitable modifications and adaptations of the variety of conditions and parameters normally encountered in in vitro assays and drug screening procedures or which are obvious to those skilled in the art are within the spirit and scope of the invention.

### EXAMPLE 1

PRIMARY SCREEN: Identifying Compounds That Activate the Caspase Cascade

| No. | Compound | EC50(nM) T47-D, 24 h |
|---|---|---|
| 1 | MX69501 | 473 |
| 2 | MX2060 | 560 |
| 3 | MX59003 | 100 |
| 4 | MX63844 | 800 |
| 5 | MX72086 | 4400 |
| 6 | MX77356 | 1800 |
| 7. | MX2105 | 90 |
| 8. | MX73936 | 360 |

The above specified compounds activate caspases in a cell line used in primary screening assay (T47D). Based on this data, all compounds would inducers of apoptosis.

### EXAMPLE 2

Secondary/tertiary Screen

Screening of apoptosis inducers for cell-cycle selectivity; Use of a cell cycle screen to identify selective compounds that are broad spectrum or cell type specific in the apoptosis inducing capabilities; Identification of selective compounds based on predictive mechanisms.

This assay is designed to determine the effect of the compound on the cell cycle, in particular, cell cycle arrest in any given stage. This assay can be used for any cell line.

Materials

12 well plate (Costar-Fisher cat. # 07-200-82)

PI solution: 1% Na Citrate, 0.1% Triton X-100, 50 ug/ml propidium iodide

FACS tubes (12×75 mm, Fisher cat. # 14-956-3A)

Procedure

1. Harvest cell line of interest and plate in 1 ml of media at 5×10⁵ cells/ml for each compound to be tested.

2. Prepare 1 mm stocks of each compound in DMSO

3. Include 0.1% DMSO control as well as 58151 control at 20 nm

4. Incubate for 0-72 hrs at 37° C. and 5% CO₂.

Cell Staining

5. Obtain and label appropriate number of FACS tubes.

6. Spin down cells at 1200 rpm for 10 min

7. Aspirate media and resuspend cells with 500 ul PI solution

8. Incubate for 30 min room temperature covered with foil

FACS analysis

9. Create a FSC vs. SSC dot plot, FL2-W vs. FL2-A dot plot, and a FL2-A histogram for control.

10. Create a FL2-A histogram for each subsequent sample

11. On the control histogram set markers to enumerate percentage of events at each stage of cell cycle.

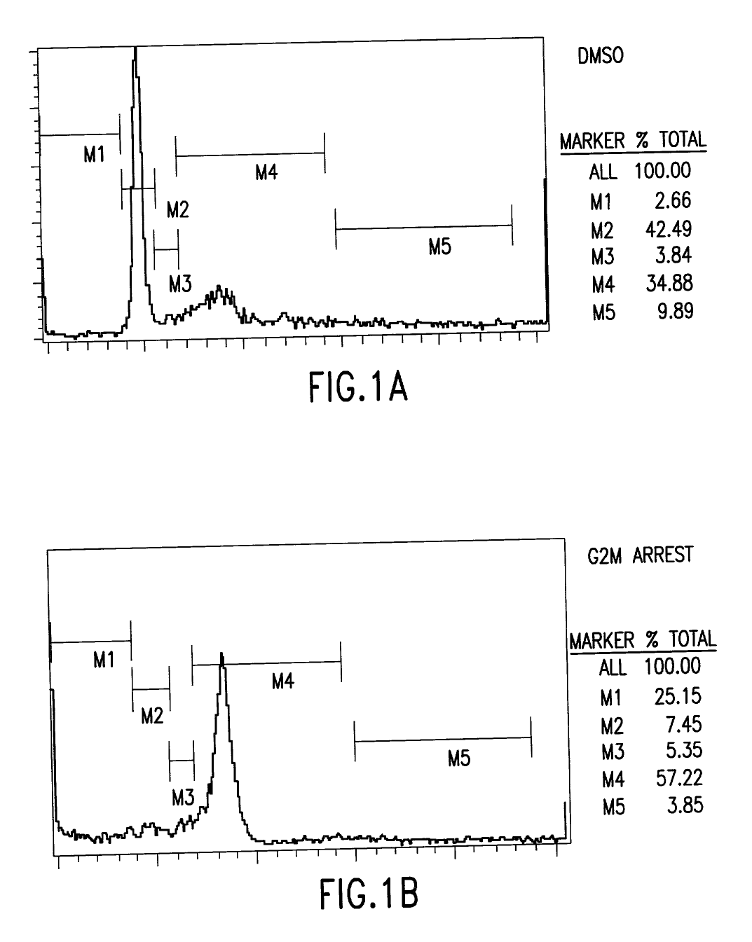

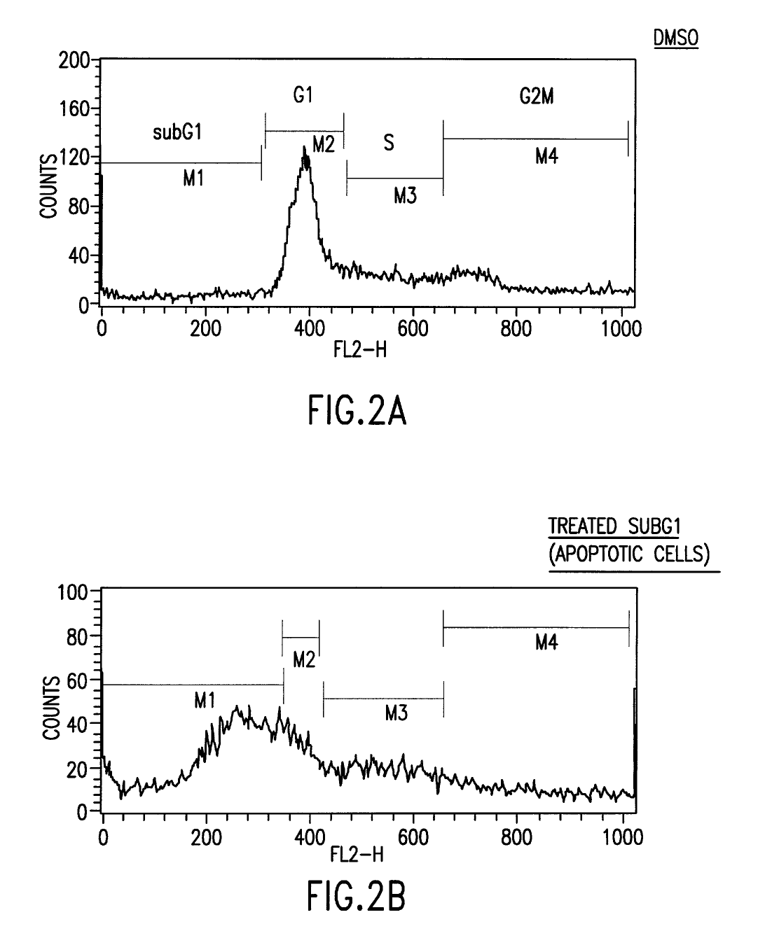

The statistics for each stage of the cell cycle are retrieved and plotted (see FIGS. 1A, 1B, 2A and 2B.

| Compound | | | Cell Cycle Block T47-D, 24 h |
|---|---|---|---|
| | 1 | MX69501 | G2M |
| | 2 | MX2060no arrest | |
| | 3 | MX59003 | G2M |
| | 4 | MX63844 | S/G2M |
| | 5 | MX72086 | G1 |
| | 6 | MX77356 | G1 |
| | 7. | MX73936 | G1 |
| | 8. | MX2105G2M | |

This analysis allows the identification of caspase inducers that are mechanistically different in their phenotype. Compounds that arrest cells in the different stages of its cell-cycle, and then induce caspases and apoptosis are predicted to have differential cytotoxic profiles in normal versus proliferating cells as well as different malignancies that often result from effects in the various cell cycle checkpoints. This assay allows the prediction and categorization of the compounds that will have selectivity in their cytotoxicity.

### EXAMPLE 3

### Determination of Cell/tissue Type Selectivity by a Caspase Assay or a Cell-type Specificity Growth Inhibition Assay Using a Diverse Cell Line Panel

A growth inhibition assay with a diverse panel of cell lines would then determine the cell-type selectivity for these compounds thereby predicting the tissue selectivity in-vivo.

T47D and ZR-75-1 cells were grown and harvested. An aliquot of 90 μl of cells (2.2×104 cells/ml) was added to a well of a 96-well microtiter plate containing 10 μl of a 10% DMSO in RPMI-1640 media solution containing 1 nM to 100 μM of the test compound (0.1 nM to 10 μM final). An aliquot of 90 μl of cells was added to a well of a 96-well microtiter plate containing 10 μl of a 10% DMSO in RPMI-1640 media solution without compound as the control sample for maximal cell proliferation (Aₘₐₓ). The samples were mixed by agitation and then incubated at 37° C. for 48 h in a 5% CO₂-95% humidity incubator. After incubation, the samples were removed from the incubator and 20 μl of CellTiter 96 AQ_{UEOUS} One Solution Cell Proliferation™ reagent (Promega) was added. The samples were mixed by agitation and incubated at 37° C. for 2-4 h in a 5% CO₂-95% humidity incubator. Using an absorbance plate reader (Model 1420 Wallac Instruments), an initial reading (T=0) was made approximately 1-2 min after addition of the solution, employing absorbance at 490 nm. This determines the possible background absorbance of the test compounds. No absorbance for the test compound was found at 490 nm. After the 2-4 h incubation, the samples were read for absorbance as above (A_{Test}).

Baseline for GI₅₀ (dose for 50% inhibition of cell proliferation) of initial cell numbers were determined by adding an aliquot of 90 μl of cells or 90 μl of media, respectively, to wells of a 96-well microtiter plate containing 10 μl of a 10% DMSO in RPMI-1640 media solution. The samples were mixed by agitation and then incubated at 37° C. for 0.5 h in a 5% CO₂-95% humidity incubator. After incubation, the samples were removed from the incubator 20 μl of CellTiter 96 AQ_{UEOUS} One Solution Cell Proliferation™ reagent (Promega) was added. The samples were mixed by agitation incubated at 37° C. for 2-4 h in a 5% CO₂-95% humidity incubator. Absorbance was read as above, (A_{Strt}) defining absorbance for initial cell number used as baseline in GI₅₀ determinations.

GI₅₀ (dose for 50% inhibition of cell proliferation) is the
concentration where [(A_{Test} − A_{Start}) / (A_{Max} − A_{Start})] = 0.5
The GI₅₀ (nM) are summarized in Table II:

| | GI₅₀(nM) | | | |
|---|---|---|---|---|
| Compound | T47-D | HeLa | PC-3 | Panc-1 |
| 59003 | 40 | >10000 | >10000 | >10000 |
| 63844 | 400 | >10000 | >10000 | >10000 |
| 2060 | 1100 | 300 | 250 | 300 |
| 73936 | 5 | 80 | 30 | >10000 |
| 72086 | 1500 | 2667 | 3000 | >10000 |
| 77356 | 350 | >10000 | >10000 | >10000 |
| 2105 | 15 | 25 | 40 | ND |
| 69501 | 175 | 300 | 210 | 400 |

Compounds like 59003, 63844 and 77356 would then be categorized as caspase inducers with different cell-cycle arrest properties but similar growth inhibition characteristics showing cell-type selectivity. These compounds are then predicted to have different target specificities. Compounds like 2105, 69501 will then be grouped as caspase inducers with similar growth arrest (G2M) and a broad range growth inhibitory properties (still selective for resting vs proliferating cells) are predicted to have similar targets. On the other hand, compounds like 2060 with no cell cycle arrest and no growth inhibition discrimination will be non-selective caspase inducers that would hit a variety of cells and predicted to have similar targets.

### EXAMPLE 4

### An In-vitro Tubulin Polymerization Assay as a Secondary Screen to Group All the Tubulin Inhibitors/stabilizers From the Primary Screen and Identify Other Targets

In order to further classify the G2/M cell cycle arrest compounds, an in vitro polymerization assay was designed.

Protocol

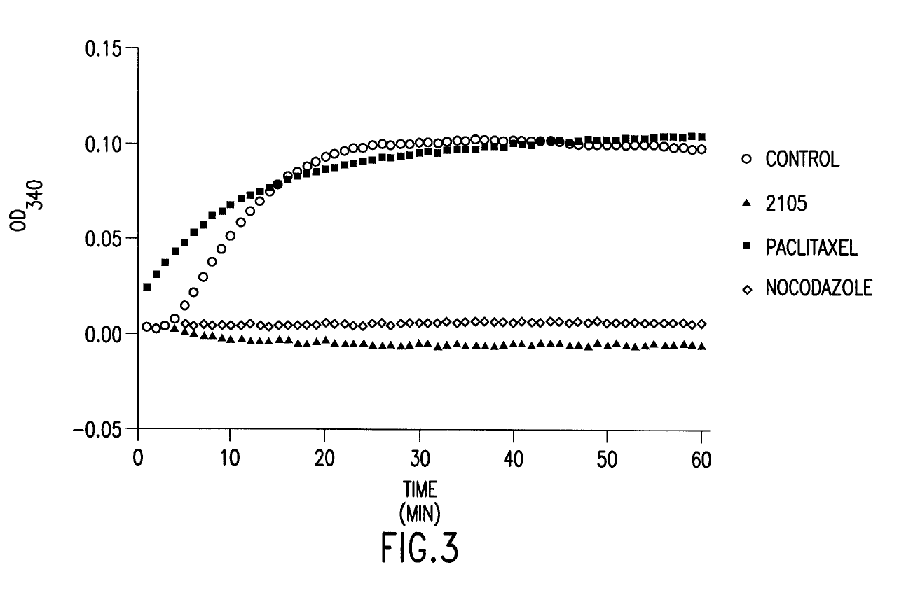

Tubulin protein (150 ug) isolated from bovine brain is resuspended in a buffer containing 80 mM PIPES, 0.5 mM magnesium chloride, 1 mM EGTA, pH 6.9 and the polymerization reaction is initiated by the addition of 1 mM GTP. The compounds are added to a final concentration of 50 uM to study the effect on the tubulin polymerization. The rate and the amount of polymerization is monitored by measuring the absorbance at 340 nm at one minutes intervals for one hour. The effect of the compound on the tubulin polymerization is determined based on the relative amount of tubulin polymerization at Vmax as compared to the DMSO control. Typically, taxol and vinblastine are included as positive controls. The results are shown in FIG. 3.

Most of the G2M compounds (80% from the primary screen) end-up being tubulin inhibitors. The cell-cycle screen followed up with an in vitro tubulin polymerization assay helped classify the G2M compounds into tubulin and non-tubulin inhibitors.

### EXAMPLE 5

### Use of a Cell-line Specific Primary Screen to Identify Compounds That Are Selective to a Particular Tumor/disease

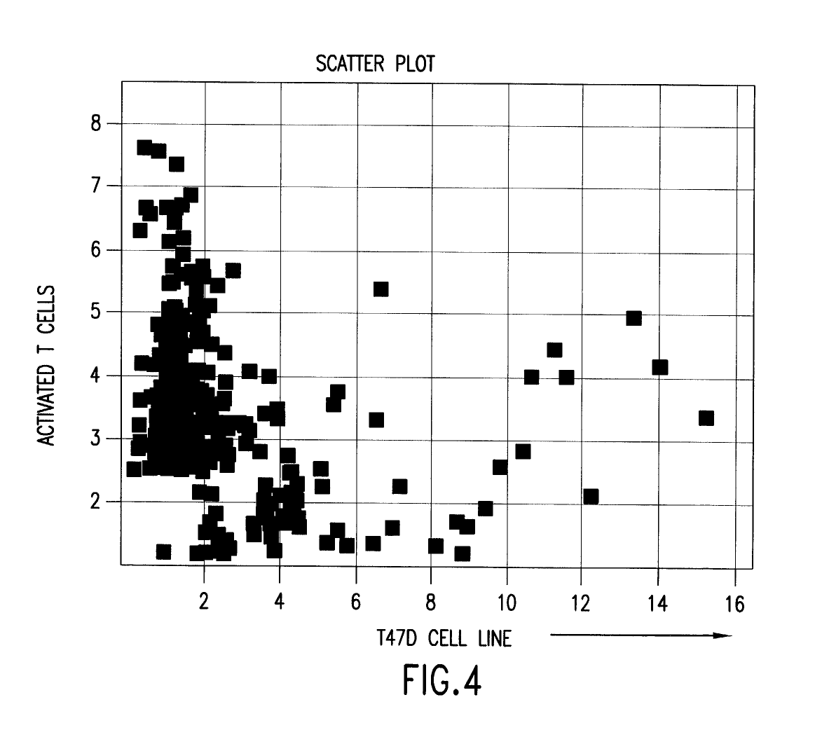

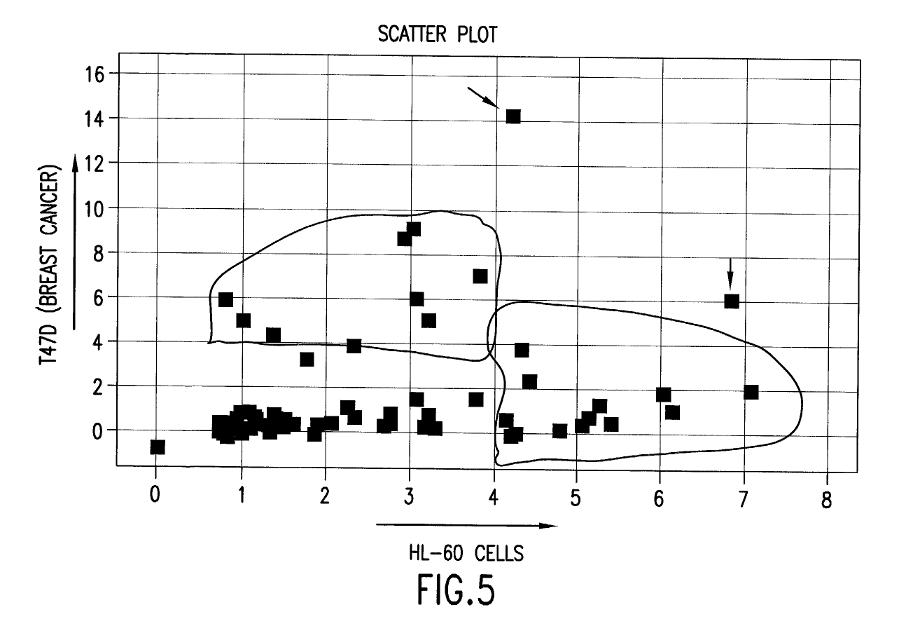

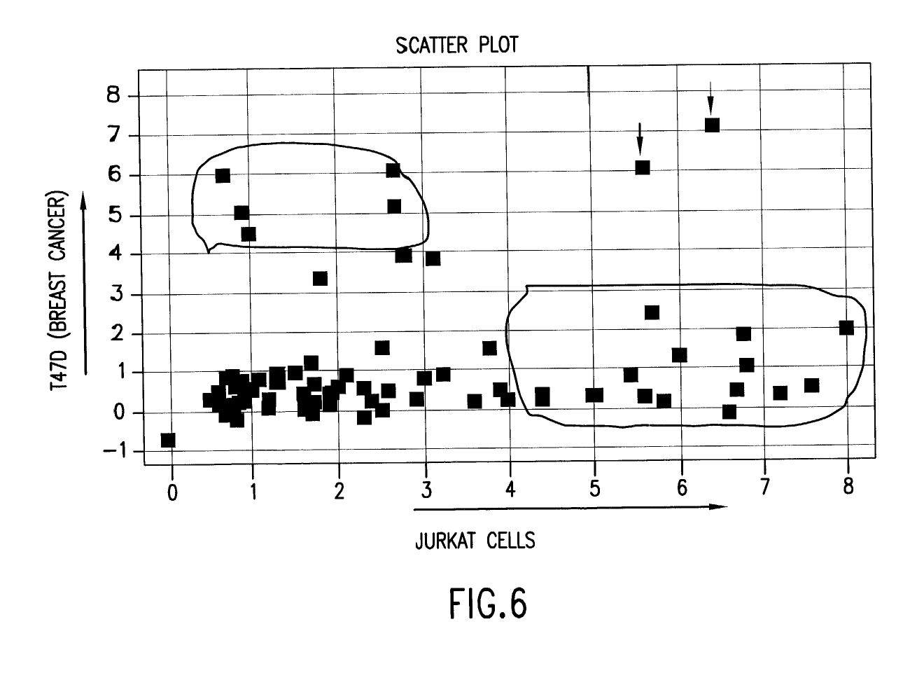

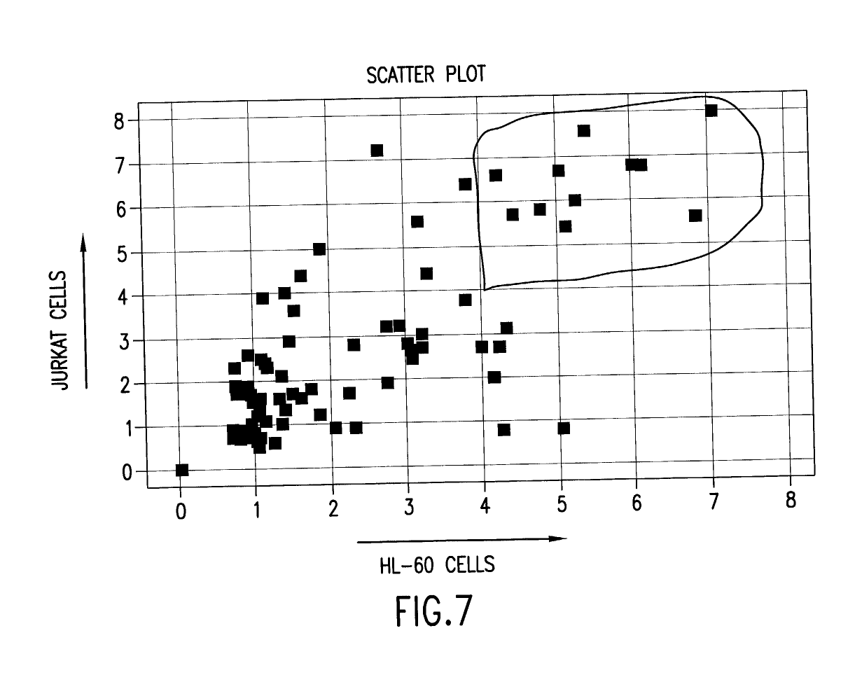

From the above screening tree, which is in practice, one could also perceive the use of a CELL-LINE specific caspase inducer screen as a primary screen to identify compounds that are selective to a particular tumor/disease. Provided here as an example is the screening assay that was done using T47D cells and activated T cells using a compound library and the comparison drawn (FIG. 4). Other examples include the differential activation of caspases using the “Killer plate” compounds in T47D (breast cancer) versus HL-60 leukemia cell line (FIG. 5) or Jurkat leukemia cell line (FIG. 6).

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of identifying potentially therapeutically effective anticancer agents by determining the ability of one or more test compounds to selectively and differentially activate the apoptosis cascade in viable cultured cancer cells having an intact cell membrane when the cells are exposed to the test compound(s) for a predetermined period of time at a predetermined temperature, wherein a test compound is determined to have potential therapeutic efficacy if said caspase cascade activity is enhanced in response to the presence of said test compound.

2. A method for identifying selective anticancer agents, comprising
determining the ability of one or more test compounds to activate the apoptosis cascade in a first viable cultured cancer cell line having an intact cell membrane when the cells are exposed to the test compound(s) for a predetermined period of time at a predetermined temperature; and
determining the ability of one or more test compounds to activate the apoptosis cascade in a second viable cultured cancer cell line having an intact cell membrane when the cells are exposed to the test compound(s) for a predetermined period of time at a predetermined temperature;
wherein a test compound is determined to be a selective anticancer agent when the caspase cascade activity is enhanced in response to the presence of said test compound in only one of the cell line.

3. A method for identifying selective anti-cancer agents comprising obtaining at least one population of viable cultured cancer cells of a first type having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; combining a second portion of the at least one population with an amount of the solvent which was used to dissolve the at least one test compound, for the predetermined period of time at the predetermined temperature thereby generating a second volume; separately adding to each of the first volume and the second volume a reporter compound having at least one measurable property which is responsive to the caspase cascade; measuring the at least one measurable property of the reporter compound in the first volume and thereby measuring the caspase cascade activity of the first volume; measuring the at least one measurable property of the reporter compound in the second volume and thereby measuring the caspase cascade activity of the second volume; calculating a first ratio of caspase cascade activity measured for the first volume to the caspase cascade activity measured for the second volume; and comparing the first ratio to at least one second ratio obtained with at least one second type of cultured cancer cells, and identifying those test compounds that have higher ratios for certain types of cultured cancer cells and are selective therefor.

4. The method of any one of claims 1-3, further comprising determining the cancer cell viability with or without the test compound.

5. A method for identifying a selective anti-cancer agent, comprising obtaining at least one population of viable cultured cancer cells having intact cell membranes from a cell growth medium under conditions conducive to growth; combining a first portion of the at least one population with a predetermined amount of at least one test compound dissolved in a solvent for a predetermined period of time at a predetermined temperature thereby generating a first volume; combining a second portion of the at least one population with an amount of the solvent which was used to dissolve the at least one test compound, for the predetermined period of time at the predetermined temperature thereby generating a second volume; separately assessing the cell viability of the first volume and the second volume; and comparing the cell viability of the first volume to the cell viability of the second volume, wherein when the cell viability of the first volume is less than the cell viability of the second volume, the at least one test compound selectively kills the cancer cells and is identified as a selective anti-cancer agent.

6. The method of claim 5, wherein cell viability is assessed by observing mitochodrial activity, membrane intactness, or cell number.

7. The method of claim 6, wherein mitochondrial activity, membrane intactness, or cell number is measured by using fluorescence methodology, colorimetric assays, or direct visualization techniques.

8. The method of claim 5, wherein cell viability is assessed by contacting the cells with a reporter compound selected from the group consisting of a fluorogenic compound that produces fluorescence under the influence changes in mitochondrial activity, membrane intactness, or cell number; a chromogenic compound that produces light absorption under the influence of changes in mitochondrial activity, membrane intactness, or cell number; and a chemiluminescent compound that produces light emission under the influence of changes in mitochondrial activity, membrane intactness, or cell number.

9. The method of any one of claims 1-3, further comprising determining the ability of the agent to arrest the cell cycle during a particular phase prior to apoptosis

10. The method of claim 9, wherein the ability of the agent to arrest the cell cycle at a particular phase is determined by combining a first portion of the cells with a predetermined amount of at least one test compound dissolved in a solvent for a predetermined period of time at a predetermined temperature thereby generating a first volume; and determining at what phase the cell cycle is arrested.

11. The method of any one of claims 1-3, further comprising determining the ability of the agent to inhibit or enhance tubulin polymerization.

12. The method of claim 11, comprising contacting a first sample of tubulin with a predetermined amount of at least one test compound dissolved in a solvent for a predetermined period of time at a predetermined temperature thereby generating a first volume; separately combining a second sample of tubulin with an amount of the solvent which was used to dissolve the at least one test compound, for the predetermined period of time at the predetermined temperature thereby generating a second volume; and comparing the extent of polymerization of the first and second samples of tubulin and thereby determining wherein the test compound inhibits or stabilizes the polymerization of tubulin.
